(19) United States
(12) Patent Application Publication
(10) Pub. No.: US 2012/0184862 A1
(43) Pub. Date: Jul. 19, 2012

(54) SYSTEM AND METHOD FOR PATIENT MONITORING

(75) Inventors: Siang Fook Foo, Singapore (SG); Jayachandran Maniyeri, Singapore (SG); Phyo Wai Aung Aung, Singapore (SG); Jit Biswas, Singapore (SG); Jianzhong Hao, Singapore (SG); Poh Leong Vincent Kng, Singapore (SG)

(73) Assignee: Agency for Science, Technology and Research, Connexis (SG)

(21) Appl. No.: 13/384,412

(22) PCT Filed: Jul. 19, 2010
(86) PCT No.: PCT/SG2010/000273

§ 371 Date: Mar. 28, 2012

Related U.S. Application Data
(60) Provisional application No. 61/226,340, filed on Jul. 17, 2009.

(51) Int. Cl. A61B 5/03; A61B 5/024; A61B 5/08; A61B 5/11

(52) U.S. Cl. 600/508; 600/561; 600/529

(57) ABSTRACT

A system and method for patient monitoring using an array of pressure sensors, and a computer readable data storage medium having stored thereon computer code means for instructing a computer to execute a method for monitoring a patient using an array of pressure sensors. The method comprising the steps of: determining a value of a selection parameter of each pressure sensor of the array; selecting one more of the pressure sensors based on the respective values of the selection parameter; and measuring a vital sign of the patient based on data obtained from said one or more selected pressure sensors.

### FIELD OF INVENTION

The present invention relates to a system and method for patient monitoring using an array of pressure sensors, and to a computer readable data storage medium having stored thereon computer code means for instructing a computer to execute a method for monitoring a patient using an array of pressure sensors.

### BACKGROUND

There is great interest in systems to automate the monitoring of patients non-intrusively. In particular, one approach is to provide an array of pressure sensors on a bed, to determine the status of the patient assigned to the bed. In such an arrangement, pressure sensors are distributed on the bed, with the sensors measuring changes in pressure.

One of the biggest challenges relate to the low signal to noise ratio when measuring certain signals such as the heart rate or respiratory rate of a patient. For example, when measuring the heart or respiratory rate of a patient, because of the low signal intensity of the heart rate when compared with the ambient noise which may result from patient movements, such heart or respiratory rate measurements are typically difficult and inaccurate.

Several approaches have been applied to overcome such difficulties in heart or respiratory rate measurements. For example, there have been disclosures of lifebed patient vigilance systems that measure heart and respiratory rates through sensor arrays and pressure switches on the clothing of the patient. Other approaches include the use of advance signal processing techniques to extract the required signals. However, there is still room for improvement, in particular with regard to the robustness and accuracy of the system.

In addition, separate systems are typically implemented to measure different parameters. For example, determining patient occupancy or movement on the bed can be implemented with a relatively inaccurate sensor, with little requirement for signal processing. On the other hand, to measure finer pressure changes as a result of heart and respiratory rates where the signal to noise ratio is relatively poor, a separate system with more accurate sensors and more signal processing is required, which may in turn be unsuitable for determining patient occupancy or movement.

Therefore, there exists a need to provide a system and method for patient monitoring that seeks to address one or more of the problems mentioned above.

### SUMMARY

In accordance with a first aspect of the present invention, there is provided a method for monitoring a patient using an array of pressure sensors, the method comprising the steps of: determining a value of a selection parameter of each pressure sensor of the array; selecting one more of the pressure sensors based on the respective values of the selection parameter; and measuring a vital sign of the patient based on data obtained from said one or more selected pressure sensors.

Determining a value of a selection parameter may comprise determining a desired sensor location; and determining a distance of each pressure sensor from the desired sensor location.

The method may comprise choosing a default pressure sensor as the selected pressure sensor when a distance between the default pressure sensor and the desired sensor location is within a threshold.

The method may comprise choosing another one of the pressure sensors as the selected pressure sensor when a distance between the default pressure sensor and the desired sensor location is outside a threshold

Determining the desired sensor location may comprise the steps of; approximating a shape of the patient based on data from the pressure sensors; and determining the desired sensor location based on the determined shape.

The method may further comprise determining a presence of the patient based on data from the pressure sensors.

Determining the presence of the patient may comprise performing one or more of a group consisting of mean, histogram, and shape analysis.

The method may further comprise determining a movement of the patient based on data from the pressure sensors.

Determining a movement of the patient on the surface may comprise one or more of a group consisting of conducting pressure point analysis using regression techniques and conducting peak detection techniques.

The vital sign may comprise heart rate or respiratory rate.

Determining the vital sign comprises one or more of a group consisting of wavelet denoising, autocorrelation and histogram techniques.

The method may further comprise analyzing the vital sign result with Pearson correlation coefficients.

The method may further comprise analyzing the vital sign result with integrated patient information or other contexts.

The method may further comprise configuring an output response in response to the vital sign result.

In accordance with a second aspect of the present invention, there is provided a system for monitoring a patient using an array of pressure sensors, the system comprising: means for determining a value of a selection parameter of each pressure sensor of the array; means for selecting one more of the pressure sensors based on the respective values of the selection parameter; and means for measuring a vital sign of the patient based on data obtained from said one or more selected pressure sensors.

The means for determining a value of a selection parameter may comprise: means for determining a desired sensor location; and means for determining a distance of each pressure sensor from the desired sensor location.

A default pressure sensor may be chosen as the selected pressure sensor when a distance between the default pressure sensor and the desired sensor location is within a threshold.

Another one of the pressure sensors may be chosen as the selected pressure sensor when a distance between the default pressure sensor and the desired sensor location is outside a threshold

The means for determining the desired sensor location may comprise; means for approximating a shape of the patient based on data from the pressure sensors; and means for determining the desired sensor location based on the determined shape.

The system may further comprise means for determining a presence of the patient based on data from the pressure sensors.

The means for determining the presence of the patient may comprise means for performing one or more of a group consisting of mean, histogram, and shape analysis.

The system may further comprise means for determining a movement of the patient based on data from the pressure sensors.

The means for determining a movement of the patient on the surface may comprise one or more of a group consisting of means for conducting pressure point analysis using regression techniques and means for conducting peak detection techniques.

The vital sign may comprise heart rate or respiratory rate.

The means for determining the vital sign may comprise one or more of a group consisting of mean for wavelet denoising, autocorrelation and histogram techniques.

The system may further comprise means for analyzing the vital sign result with Pearson correlation coefficients.

The system may further comprise means for analyzing the vital sign result with integrated patient information or other contexts.

The system may further comprise means for configuring an output response in response to the vital sign result.

In accordance with a third aspect of the present invention, there is provided a computer readable data storage medium having stored thereon computer code means for instructing a computer to execute a method for monitoring a patient using an array of pressure sensors, the method comprising the steps of: determining a value of a selection parameter of each pressure sensor of the array; selecting one more of the pressure sensors based on the respective values of the selection parameter; and measuring a vital sign of the patient based on data obtained from said one or more selected pressure sensors.

### BRIEF DESCRIPTION OF THE DRAWINGS

Embodiments of the invention will be better understood and readily apparent to one of ordinary skill in the art from the following written description, by way of example only, and in conjunction with the drawings, in which:

FIG. 1 shows an example embodiment of an FBG sensor data system.

FIG. 2 shows an example embodiment of an FBG sensor data system implemented for monitoring of a single bed.

FIG. 3 shows an example embodiment of an interrogator unit.

FIG. 4 shows an example embodiment of a sensor array layout.

FIG. 5 shows an example deployment of a sensor array on a bed.

FIG. 6 shows an example sensor system monitoring a plurality of beds.

FIG. 7 is a flow chart illustrating a method for monitoring of respiratory, heart rate, pressure points and occupancy of a patient on a bed, implemented in an example embodiment.

FIG. 8 shows screenshots of a program for graphically representing and displaying the movement of a patient, implemented in an example embodiment.

FIG. 9 is a graph illustrating peak detection implemented in an example embodiment.

FIG. 10a shows a patient lying on a bed in the supine position in an example embodiment.

FIG. 10b shows a patient lying on a bed in the recumbent position in an example embodiment.

FIG. 10c shows an example embodiment of the shape of a person determined by the linear regression technique, when the person is lying diagonally across the bed.

FIG. 11 is a block diagram illustrating the decomposition process implemented in an example embodiment.

FIG. 12 depicts a normalized respiratory signal with no body movements obtained from a sensor in an example embodiment.

FIG. 13 shows a signal convoluted with the low-pass filter in an example embodiment.

FIG. 14 shows the signal after undergoing high-pass filtering in an example embodiment.

FIG. 15 shows a sample output after undergoing the autocorrelation function in an example embodiment.

FIG. 16 shows a histogram of periodic components obtained from an example embodiment.

FIG. 17 shows another example embodiment of the present invention for deployment in a hospital ward.

FIG. 18 shows an example embodiment of a sensor array of sensors paced on top of a mattress.

FIG. 19a-19c shows an example implementation of the sensor array on a mattress.

FIG. 20 shows an example embodiment of an adjustable bed frame.

FIG. 21 shows a snapshot of automated pressure profile and occupancy monitoring provided by an example embodiment of the present invention.

FIG. 22 shows a snapshot of automated respiratory and pulse rate monitoring provided by an example embodiment of the present invention.

FIG. 23 shows historical and trend charts of pulse rates, respiratory rates and occupancy provided by an example embodiment of the present invention.

FIG. 24 is a schematic diagram of the method and system of the example embodiment implemented on a computer system.

FIG. 25 is a schematic diagram of the method and system of the example embodiment implemented on a wireless device.

FIG. 26 is a flow chart illustrating a method of patient monitoring using an array of pressure sensors in an example embodiment.

### DETAILED DESCRIPTION

Embodiments of the present invention seek to provide a method and system for continuously monitoring the health status of patients on their respective beds, in a non-intrusive manner. The bed comprises an array of sensors for detecting pressure changes, with each sensor connected to interrogators which collect the data obtained at each sensor. Processing units then analyse the data obtained by the interrogators. Based on the analysis, a desired set of sensors is selected to determine a variety of parameters indicative of the health status of the patients.

Some portions of the description which follows are explicitly or implicitly presented in terms of algorithms and functional or symbolic representations of operations on data within a computer memory. These algorithmic descriptions and functional or symbolic representations are the means used by those skilled in the data processing arts to convey most effectively the substance of their work to others skilled in the art. An algorithm is here, and generally, conceived to be a self-consistent sequence of steps leading to a desired result. The steps are those requiring physical manipulations of physical quantities, such as electrical, magnetic or optical signals capable of being stored, transferred, combined, compared, and otherwise manipulated.

Unless specifically stated otherwise, and as apparent from the following, it will be appreciated that throughout the present specification, discussions utilizing terms such as “computing”, “calculating”, “determining”, “selecting”, “generating”, “analyzing”, “configuring”, or the like, refer to the action and processes of a computer system, or similar electronic device, that manipulates and transforms data represented as physical quantities within the computer system into other data similarly represented as physical quantities within the computer system or other information storage, transmission or display devices.

The present specification also discloses apparatus for performing the operations of the methods. Such apparatus may be specially constructed for the required purposes, or may comprise a general purpose computer or other device selectively activated or reconfigured by a computer program stored in the computer. The algorithms and displays presented herein are not inherently related to any particular computer or other apparatus. Various general purpose machines may be used with programs in accordance with the teachings herein. Alternatively, the construction of more specialized apparatus to perform the required method steps may be appropriate. The structure of a conventional general purpose computer will appear from the description below.

In addition, the present specification also implicitly discloses a computer program, in that it would be apparent to the person skilled in the art that the individual steps of the method described herein may be put into effect by computer code. The computer program is not intended to be limited to any particular programming language and implementation thereof. It will be appreciated that a variety of programming languages and coding thereof may be used to implement the teachings of the disclosure contained herein. Moreover, the computer program is not intended to be limited to any particular control flow. There are many other variants of the computer program, which can use different control flows without departing from the spirit or scope of the invention.

Furthermore, one or more of the steps of the computer program may be performed in parallel rather than sequentially. Such a computer program may be stored on any computer readable medium. The computer readable medium may include storage devices such as magnetic or optical disks, memory chips, or other storage devices suitable for interfacing with a general purpose computer. The computer readable medium may also include a hard-wired medium such as exemplified in the Internet system, or wireless medium such as exemplified in the GSM mobile telephone system. The computer program when loaded and executed on such a general-purpose computer effectively results in an apparatus that implements the steps of the preferred method.

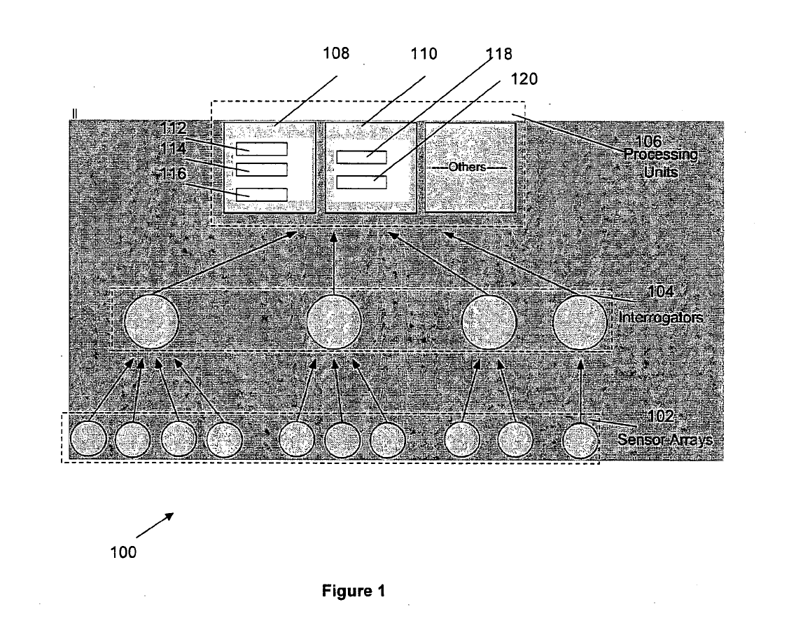

FIG. 1 shows an example embodiment of an FBG sensor data system 100 deployed in e.g. a hospital ward of a plurality of patient beds. The system 100 has a distributed hierarchical topology, wherein continuous streams of observations from a plurality of sensor arrays 102, each of which comprises a plurality of sensors placed on a single bed, are collected at a corresponding interrogator e.g. 104. Sensor data from the interrogators 104 are then forwarded to a Processing Unit 106. The Processing Unit 106 comprises of a Sensor Selection and Configuration Unit 108, Analyser Unit 110 and other units. At the Processing Unit 106, sensor management processes are executed and sensor observations obtained from the interrogators 104 are analysed and processed.

The sensor selection and configuration unit 108 comprises of a sensor data acquisition module 112 for coordinating the client programs and the interrogator e.g. 104, a sensor selection module 114 for selecting the appropriate sensor for interrogation, depending on the situation, and a sensor configuration module 116 for receiving feedback from the analyser unit 110 and for configuring the sensors accordingly. The analyzer unit 110 comprises the monitoring modules 118 for all the algorithms to perform e.g. heart rate monitoring, respiratory rate monitoring, pressure points monitoring and occupancy monitoring. The analyser unit 110 also comprises a data visualization module 120 which allows the data obtained from the sensors and its derived parameters to be graphically displayed on a display unit, such as a monitor, which may be connected to the processing unit.

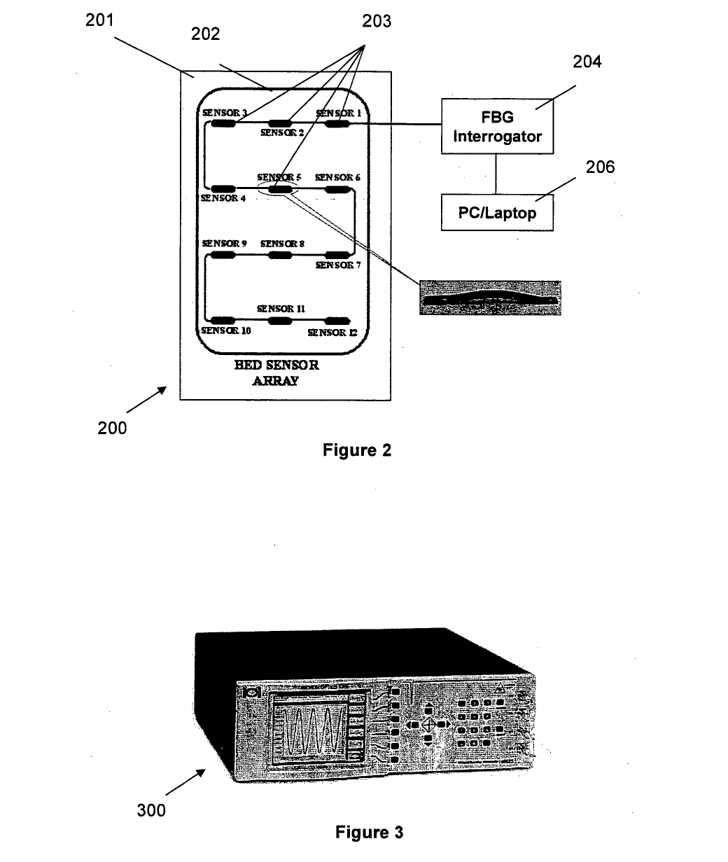

FIG. 2 shows an example embodiment of an FBG sensor data system 200 implemented for monitoring of a single bed. The sensor data system 200 is a simplified version of the system 100 illustrated in FIG. 1. Instead of monitoring a plurality of beds, only a single bed 201 is monitored. As such, there is only one sensor array 202 comprising a plurality of sensors e.g. 203. The sensors e.g. 203 are connected in series to an FBG interrogator 204 which is in turn coupled to a PC/Laptop 206. It will be appreciated that the PC/Laptop 206 functions like the processing unit 106 of FIG. 1, wherein the PC/Laptop 206 manages the operations of the sensors 203 in the sensor array 202, and analyses the data obtained from the interrogator 204.

FBG sensors e.g. 203 are understood by a person skilled in the art and are not described in detail here. A description of FBG sensors may be found in the PCT application, PCT/SG2006/000086: “Fiber Bragg Grating Sensor”, the contents of which are incorporated herein by reference. Further, it will also be understood that other types of e.g. sensors may used in place of the FBG sensors.

FIG. 3 shows an example embodiment of an interrogator unit 300. The interrogator unit 300 performs center wavelength measurements on the optical sensors e.g. 203 (FIG. 2). Powered by a high output power swept laser, the interrogators are capable of performing simultaneous measurements on hundreds of sensors repetitively within a second. Depending on the channel expansion module used, the total sensor count can be further increased. To prevent any potential data loss, the interrogator units can maintain internal data buffers of backdated wavelength data sets. The interrogator unit 300 can be controlled and monitored remotely through an extensive set of Ethernet controls and commands.

In the example embodiments, a “command and response” approach is adopted for the wavelength data acquisition from the interrogators. A data requesting command is sent from the client PC e.g. the processing unit 106 (FIG. 1) or the PC/Laptop 206 (FIG. 2) to the interrogator e.g. 300 (FIG. 3), which then triggers a data transfer back to the client. This method is used for most of the client-to-interrogator communication in the example embodiments. In other interrogators in alternative embodiments, e.g. data streaming modes are supported. The data streaming mode can reduce the overall communication overhead which in turn alleviates the transmission load of large amounts of data from the interrogators to the client.

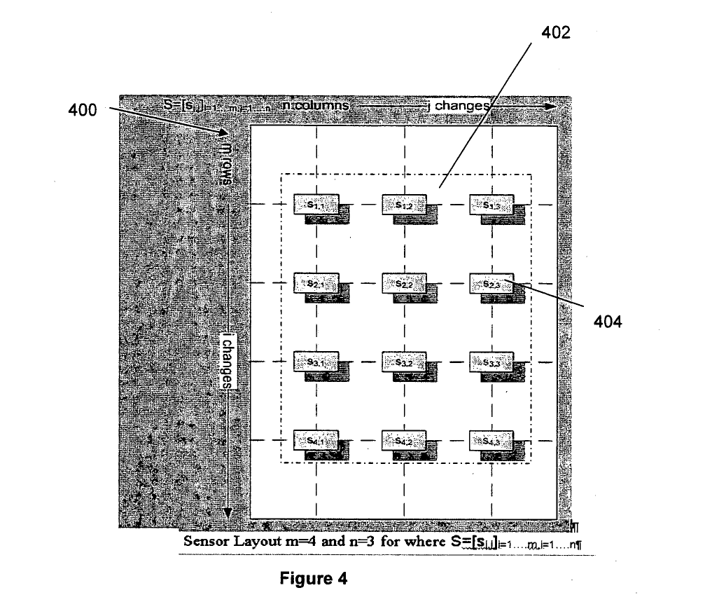

FIG. 4 shows an example embodiment of a sensor array layout 400 for a sensor array for monitoring a single bed. The sensors 402 are arranged in a 2-dimensional n×m matrix, with n representing the number of columns and m representing the number of rows. In the example embodiment, there are a total of 12 sensors e.g. 402a arranged in 3 evenly spaced columns and 4 evenly spaced rows. For ease of identifying a particular sensor with the array, each sensor may be denoted by its column and row number. For example, the sensor 402a lies in the third column and second row of the 2-D matrix, and is therefore identified as sensor S_{2,3}.

Each of the sensors 402a are pressure sensors which provide a continuous value of amplitude phase shifts in accordance with the pressure detected by the sensor. In the example embodiment, the amplitude of phase shifts can be divided into 256 different levels. As such, the sensors are capable of discerning 256 different levels of pressure. Further, the sensors 302 are controlled by the sensor configuration unit 108 (FIG. 1) to sample at a rate of 25 Hz. Therefore, given a total of 12 sensors in the sensor array, the processing unit 106 of FIG. 1 can receive a total of 3000 readings from the sensors e.g. 302a, over a period of 10 seconds. FIG. 5 shows an actual deployment of the sensor array 500 on a frame of the bed.

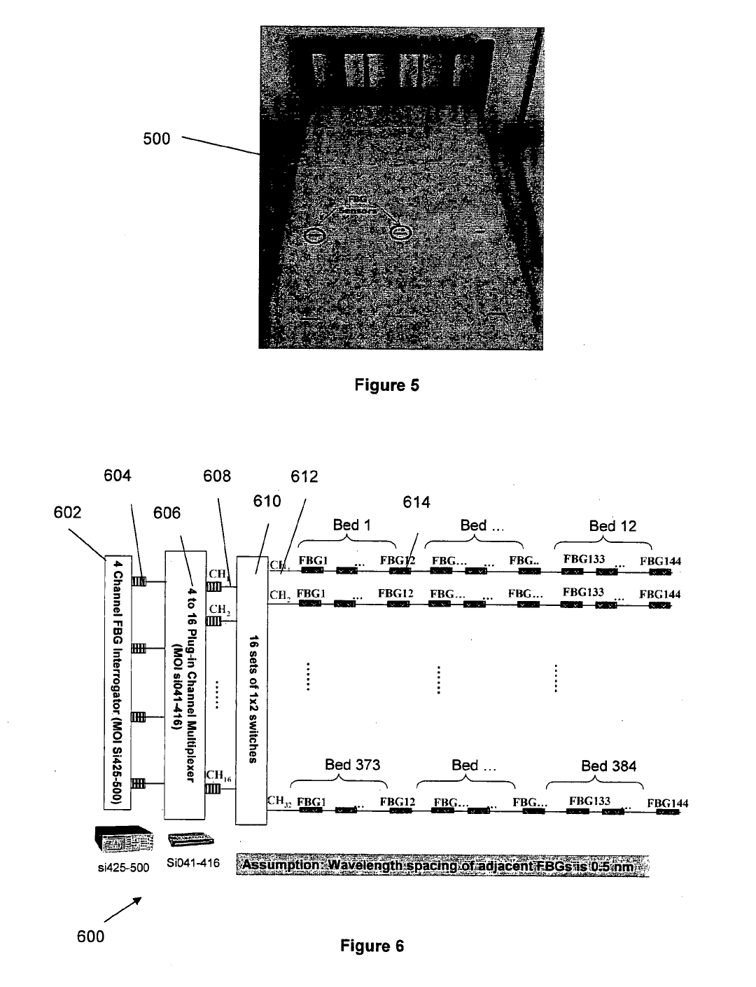

FIG. 6 shows an example sensor system 600 monitoring a plurality of beds, wherein the system has been extended using switches. As shown in FIG. 6, the sensor system 600 comprises a 4-channel FBG interrogator 602 which is capable of interrogating 4 different channels e.g. 604 at a given time. A 4-to-16 channel multiplexer 606 is coupled to the 4-channel FBG interrogator 602 to multiplex the number of channels interrogated by the 4-channel FBG interrogator 602 to 16 multiplexed channels e.g. 608. Further, 16 sets of 1×2 switches 610 are coupled to the 16 multiplexed channels e.g. 608, to double the total number of sensor channels e.g. 512 to 32. 144 FBG sensors, e.g. 614, are coupled to each sensor channel e.g. 612 serially. In the example embodiment, as 12 sensors e.g. 614 are used to monitor each bed, a total of 12 beds may be monitored on each sensor channel e.g. 612. Given that there are 32 sensor channels, a total of 384 beds may be monitored concurrently. It will be appreciated by a person skilled in the art that at any given time, the interrogator 602 interrogates a first set of 4 different sensor channels e.g. channels 1, 9, 17 and 25. Once sufficient time has lapsed for the successful interrogation of the first 4 sensor channels e.g. channels 1, 9, 17 and 25, the multiplexer 606 and switches 610 select to a different set of 4 sensor channels e.g. 2, 10, 18 and 26 for interrogation by the interrogator 602. Over time, all sensor channels e.g. channels 1 to 32 are interrogated, and the cycle is repeated, the first 4 sensor channels being interrogated again.

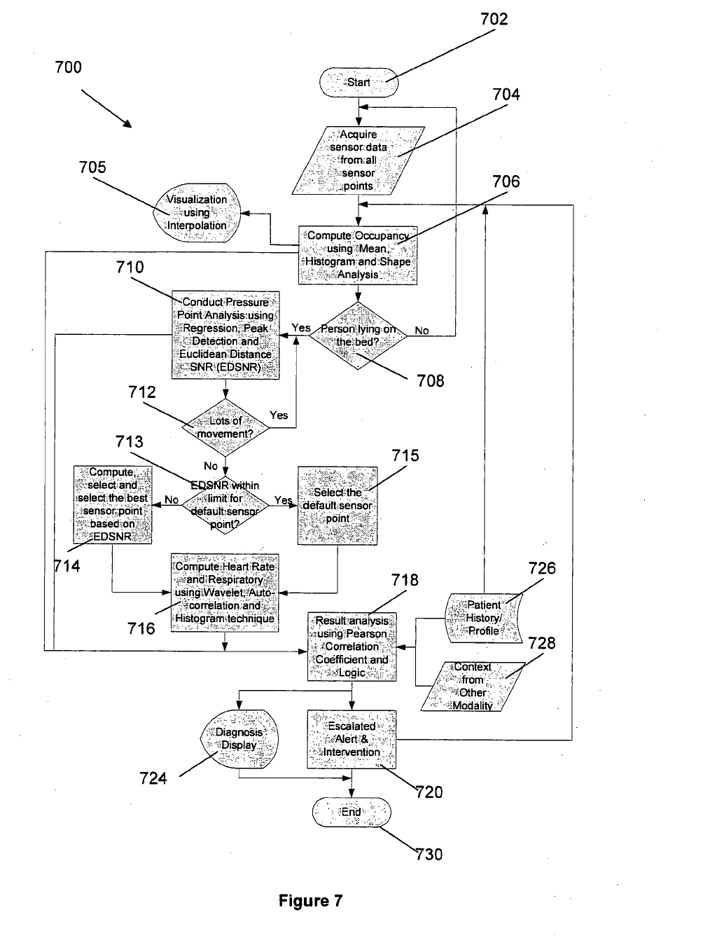

FIG. 7 is a flow chart 700 illustrating a method for monitoring of respiratory, heart rate, pressure points and occupancy of a patient on a bed, implemented in an example embodiment. The method first begins at step 702. At step 704, sensor data is acquired from each sensor e.g. 203 (FIG. 2) of the sensor array e.g. 102 (FIG. 1) via the interrogators e.g. 104 (FIG. 1), at the processing unit e.g. 106.

At step 706, based on the sensor data obtained at step 704, the monitoring module 118 of the processing unit 106 (FIG. 1) performs mean, histogram and shape analysis to determine the three parameters of mean, histogram and shape. These three parameters can be used to determine the occupancy of the bed, i.e. if there is a person lying down on the bed, at step 708.

To calculate the mean parameter, mean analysis is performed wherein the mean of all sensor data readings on the bed are calculated using the following equation:

$$\overline{x} = \frac{1}{n} {\sum }_{i = 1}^{n} {x}_{i}$$

For histogram analysis, a histogram is computed to map and count the number of observations that fall into the different and disjoint categories of sensor data readings.

For shape analysis, a string matching technique is used where the algorithm seeks to determine if the presently detected sensor readings match a set representative of a person lying on the bed.

Suppose that two region boundaries, A and B, are coded into strings denoted a₁ a₂ . . . aₙ and b₁ b₂ . . . bm, respectively. A can refer to a template pressure profile while B can refer to the actual pressure profile obtained by the sensors. Let M represent the number of matches between the two strings, where a match occurs in the kth position if aₖ=bₖ. The number of symbols that do not match is

Q=max(|A|,|B|)−M

where |arg| is the length (number of symbols) in the string representation of the argument. Q will be equal to 0 if and only if A and B are identical. The similarity between A and B is measured by the ratio

$$R = \frac{M}{Q} = \frac{M}{max ( \vert A \vert , \vert B \vert ) - M}$$

Hence R is infinite for a perfect match and 0 when none of the symbols in A and B match (M=0 in this case).

With the mean, histogram and shape analysis performed in step 706, visualization using interpolation can be performed at step 705 (compare also visualisation module 120 in FIG. 1.

Based on the mean, histogram and shape analysis in step 706, the bed occupancy (i.e. whether the person is lying on the bed) can be determined at step 708 should they exceed their respective threshold values. If it is determined that there is nobody lying on the bed, the method returns to step 704.

If it is determined that there is a person lying on the bed, the method proceeds to step 710. At step 710, pressure point analysis is conducted by the analyser unit 110 (FIG. 1) using linear regression, peak detection and Euclidean distance SNR.

The linear regression approach can identify large patient movements, such as total body movements, on the bed. The previously calculated mean of the sensor readings are used. The coordinates of sensors with readings exceeding the mean are plotted on a 2-Dimensional XY graph. It will be appreciated that the sensitivity of the points can be tuned by adjusting the threshold reading level. For example, for reduced sensitivity, the threshold reading level can be adjusted such that only sensors with readings exceeding e.g. a multiple of the mean value are plotted.

With the plotted points, a linear regression with the following equation:

$$m = \frac{n \sum ( xy ) - \sum x \sum y}{n \sum ( {x}^{2} ) - {( \sum x )}^{2}} ,$$

where n is the number of samples, x,y are the corresponding coordinates,

is performed to obtain a line of best fit from the set of plotted data points. The gradient of the obtained line is then calculated. As the patient moves on the bed, a different set of plotted points result and a new line of best fit is obtained. By detecting the change in the gradient value, the magnitude of the movement made by the patient can be calculated.

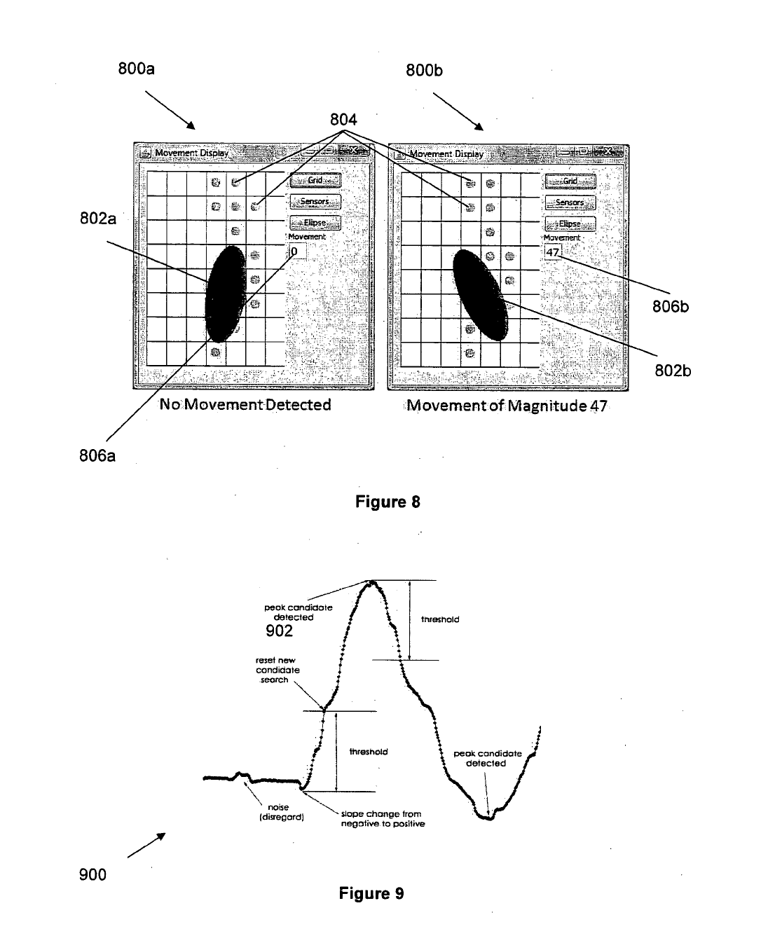

FIG. 8 shows screenshots 800a, 800b of a program written to graphically represent and display the movement of a patient, implemented in an example embodiment. The ellipses 802a, 802b give visual indications of the result of the linear regression. The points e.g. 804 displayed are the points relevant to the linear regression calculation. The movement value shown 806a, 806b gives an indication of the magnitude of movement made by the patient compared to the previous time instance. In this regard, screenshot 800a shows a display where no movement is detected, while screenshot 800b shows a display where a movement is detected.

Another approach for detecting big user's movement is the Centre of Pressure (COP) using Peak Detection. This approach is based on the COP of the patient's weight on the sensor array. The motion of patient's body can be seen as a function of the motion of the centre of pressure. As a first step, moments of each pressure element are summed and divided by the total pressure on the bed at that moment. This result is referred to as the Center of Pressure (COP) and is related to the position of the patient's center of pressure as a proportion of the distance from a reference point on the bed. In the example embodiments, the reference is taken as centre of the bed, both horizontally and vertically. The sensor pressure readings are assigned weights to find the COP along the rows and columns. Thus, the COP gives the distance of the patient's weight from the reference. As the COP of the rows and columns may not provide smooth signals and may be noisy, a Butterworth digital filter can be implemented to filter the signal obtained from the centre of pressure along rows and columns, as the frequency response of the filter is maximally flat in the passband. The bandwidth can be chosen based on practical considerations. In the example embodiments, Butterworth coefficients were found with order of filter N=2 and cut-off frequency=0.015 Hz.

In the example embodiments, it was observed that the occurrence of any movement of the patient changes the value of the centre of pressure so that it produces a peak 902 in the signal 900 as shown in FIG. 9. A peak detection algorithm is implemented that locates possible positive or negative peaks, and is controlled by a threshold value. The threshold value dictates the degree of “peakiness” that is allowed for a local maximum to be considered a “genuine” peak resulting from movements. Based on peak detection algorithm of the COP along the rows and columns, example embodiments can classify the patient as moving from side to side (left to right or right to left), sitting up or lying down with a degree of accuracy.

Based on the pressure point analysis using regression and peak detection techniques above, the example embodiments can then determine if there are movements which are large enough to make the determination of vital sign monitoring difficult. Step 712 of the method shown in FIG. 7 illustrates this. If it is determined, based on regression and/or peak detection techniques that the patient is moving about on the bed, the method returns to step 710, wherein pressure point analysis using regression and/or peak detection techniques are repeated until it can be determined that the patient is not moving about.

EDSNR (Euclidean Distance SNR) for heart and respiratory rate monitoring of each sensor is also determined at step 710. In the example embodiments, dynamic sensor selection and configuration for heart and respiratory rate monitoring is based on the Euclidean Distance SNRs. The Euclidean Distance of each sensor is the distance between the sensor and the estimated ideal sensor Iodation for monitoring a particular vital sign e.g. heart rate or respiratory rate. EDSNR is defined as the inverse of the Euclidean Distance. Hence, when the Euclidean Distance is equal to zero, EDSNR will be infinity. The purpose of the sensor selection and configuration process is to select an optimal set of sensors or sensor which can provide data of sufficient quality to perform the monitoring of vital signs.

If it is determined at step 712, that the patient is not moving about, the method proceeds to step 713. At step 713, the EDSNR of a default sensor is compared with a threshold limit. If it is determined that the EDSNR of the default sensor is within the limit, the default sensor is selected at step 715 as the monitoring sensor and the method proceeds to step 716. If it is determined that the EDSNR of the default sensor is not within the limit, a “best” sensor will be selected at step 714. At step 714, the selected sensor will be the sensor with the minimum Euclidean Distance from the ideal sensor location. As such, the sensor selected for performing the actual monitoring is also the sensor at the maximum EDSNR from the ideal sensor.

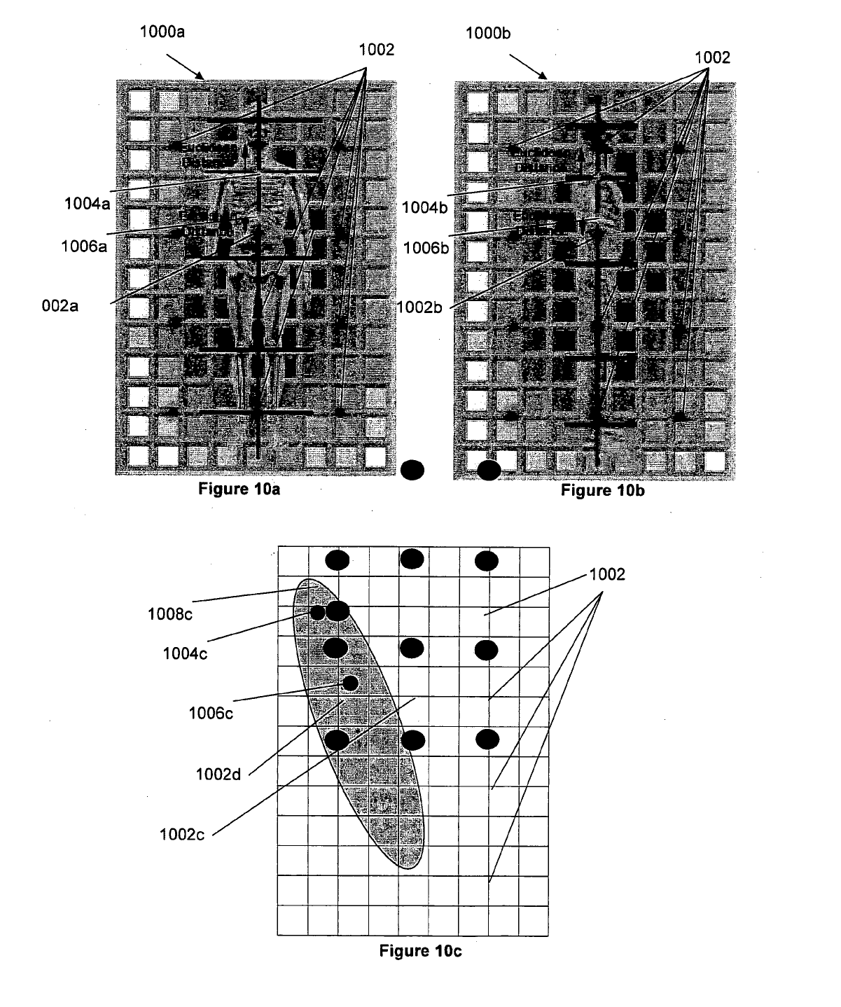

FIGS. 10a and 10b shows example sleeping postures of a patient lying on a bed with an implementation of the example embodiment. FIG. 10a shows a patient lying on a bed in the supine position, while FIG. 10b shows a patient lying on a bed in the recumbent position. As shown in the FIGS. 10a and 10b, the dots e.g. 1002 represent the placement positions of calibrated sensors. Ideal sensor locations for monitoring for the heart rate are marked with reference numerals 1004a, 1004b, in FIGS. 10a and 10b respectively. Ideal sensor locations for performing respiratory rate monitoring are marked 1006a, 1006b in FIGS. 10a and 10b respectively. The ideal sensor locations e.g. 1004a, 1004b, 1006a and 1006b are calculated using data from the regression technique earlier performed in step 710 (FIG. 7) to determine the posture of the patient presently on the bed. The proportions of the patient are estimated with a model of the Vitruvian Man by Leonardo Da Vinci. In this regard, Euclidean distances between each sensor e.g. 1002 and the ideal sensor position 1006a, 1006b, for e.g. monitoring respiratory rate and/or heart rates are computed. As seen in FIGS. 10a and 10b, the selected sensor for respiratory rate monitoring is 1002a and 1002b respectively. Sensors 1002a and 1002b may be the default sensors, described in step 713 of FIG. 7, for measuring respiratory rate. As such, since the EDSNR of the default sensors 1002a, 1002b are within a threshold EDSNR, they are selected at step 714 of FIG. 7 as the respective sensors for respiratory rate monitoring.

FIG. 10c shows an example embodiment of the shape 1008c of a person determined by the linear regression technique performed in step 710, when the person is lying diagonally across the bed. The shape 1008c is representative of the body of the person lying on the bed. Ideal sensor locations for monitoring the heart and respiratory rates are marked 1004c and 1006c respectively. In this example embodiment, the default sensor for monitoring respiratory rates is referenced by reference numeral 1002c. However, as the EDSNR of the default sensor 1002c is beyond the threshold limit, a “best” sensor is to be computed, as described in step 714 of FIG. 7. In the example embodiment, the “best” sensor based on EDSNR is determined to be sensor 1002d. Sensor 1002d is hence selected as the sensor for measuring respiratory rates.

In example embodiments, more than one sensor may be selected for measurements. For example, in instances where EDSNR of two sensors are identical, the two sensors may be selected for measurements. Alternatively, in addition to the selected “best” sensor, sensors neighbouring the selected “best” sensor may also be selected for measurement.

In example embodiments, experimental data has shown that even in scenarios where the calibration of sensors cannot be done accurately, selecting sensors for heart rate monitoring and/respiratory rate monitoring based on ESDNR can improve the overall monitoring performance.

Returning to FIG. 7, at step 716, with the sensors for respectively performing heart and respiratory monitoring identified, the heart rate and respiratory rate can be computed using wavelet denoising, auto-correlation and histogram techniques.

In the wavelet denoising technique employed in the example embodiments, with the knowledge of reference wavelengths for each FBG sensor, wavelength data received from the FBG sensors are mapped into pressure change signals. This data can be received in real-time from the interrogator and can be processed without much delay to derive the respiratory rate or heart rate of the person lying in bed. The pressure change signals are in time domain, which can be represented as signal intensity changes as a function of time. The signal, if plotted, will have axes of time and amplitude, which results in a time-amplitude representation of the signal. Such representation does not provide much useful information about the signal. Mathematical transformations are required to extract further information that is not readily available from this raw signal. The signal received has components related to respiratory movements, movements caused by the heart e.g. the pulse, and components related to other movements of the patient in bed. To calculate respiratory rate, heart rate and to plot the signals, the desired monitoring signals have to be separated from movement related signals.

One approach will be to use bandpass filtering and to detect the peak/significant frequencies based on fourier transform. This approach can be effective if the frequency band of the desired signal is easily separable from the frequency band of unwanted signals. The normal respiratory rate can be in the range of 10-30 beats per minute and pulse rate in the range of 40-120 beats per minute. Unfortunately, movement-related frequency spectrum overlaps with that of the expected respiratory rate signal and pulse rate signal frequency bands, and this makes the separation rather difficult using simple bandpass filtering. Further, as the signal intensity of the desired respiratory rate and pulse rate signals are typically weaker than the movement related signals, the difficulty of the separation process is increased.

Fourier transform has a further limitation of time-frequency resolution. For processing of continuous real-time signals, usually STFT (Short Time Fourier Transform) is applied where the continuous stream of signal is first windowed into a signal of finite length. Fourier transform is then applied to this finite length signal to detect the relevant frequency components. If the window is too short, frequency information can be modified unintentionally. If the window is too large and if the signal (respiratory or pulse) rate changes within this period, the rate change will not be visible in the result.

Embodiments of the present invention apply wavelet principles, wherein the time-frequency resolution and separation of desired signal can be improved significantly. For a practical approach to wavelet transformation, wavelet computations are performed at discrete scales, referred to as Discrete Wavelet Transform (DVVT). Based on DWT a signal (with noise) can be broken down to different components based on their scales. For the DWT computation, the discrete time-domain signal is passed through successive low-pass and high-pass filters. Such a methodology will be appreciated by a person skilled in the art to be a Mallat-tree decomposition.

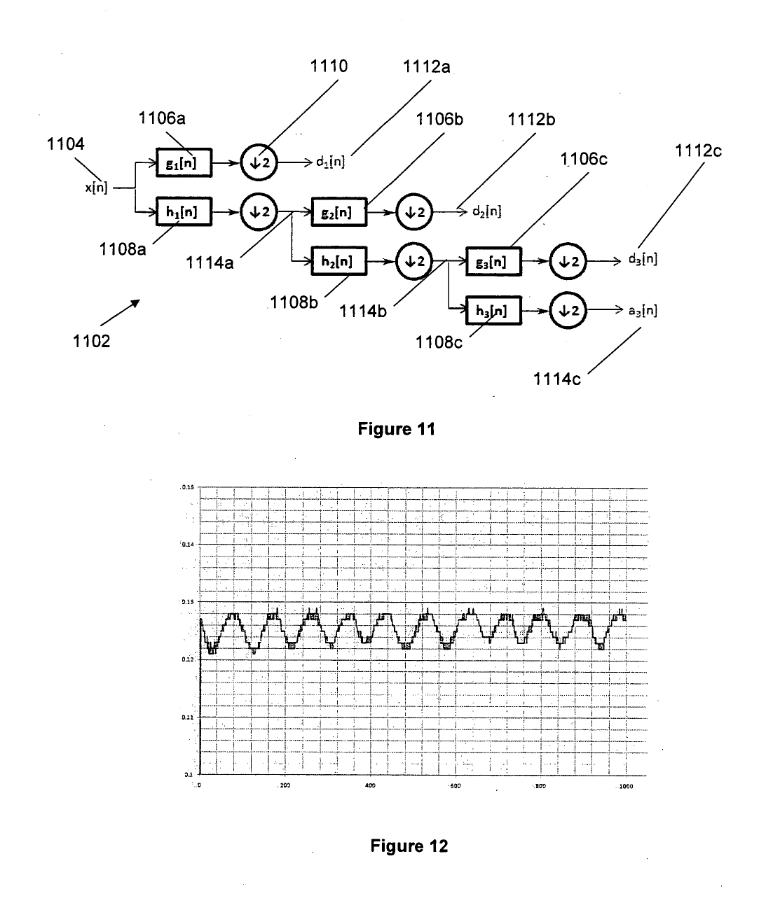

FIG. 11 is a block diagram illustrating the decomposition process 1102 implemented in an example embodiment. x[n] 1104 is the signal obtained from the selected sensor to be analysed. g[n] 1106a, 1106b, 1106c each represent high pass filtering processes, h[n] 1108a, 1108b, 1108c each represent low pass filtering processes and “↓2”, e.g. 1110 each represent a sub-sampling process. At each level, the high-pass filtering processes 1108a, 1108b, 1108c produce detailed information d[n] 1112a, 1112b, 1112c, while the low-pass filtering processes 1106a, 1106b, 1106c produce coarse approximations a[n] 1114a, 1114b, 1114c. With every level of decomposition, the detailed part or the higher frequency components 1112a, 1112b, 1112c, are separated from the approximation or low frequency components 1114a, 1114b, 1114c. Approximation parts 1114a, 1114b, 1114c are further decomposed to remove the high frequency noise. The decomposition process can be repeated until desired signal can be separated from the rest of the unwanted signals. In the example embodiments, quadratic spline wavelets are used to perform the decomposition and to separate respiratory and pulse signals from unwanted noise.

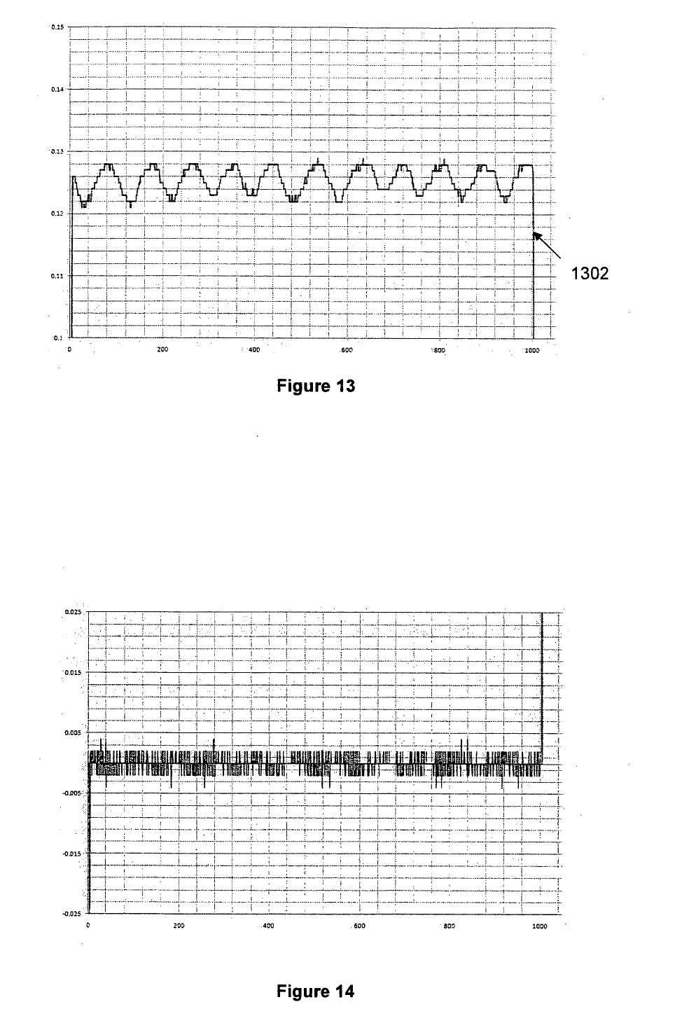

FIGS. 12-16 illustrate the wavelet decomposition process implemented in an example embodiment of the present invention. FIG. 12 depicts a normalized respiratory signal with no body movements obtained from a sensor in an example embodiment. The respiratory signal is then convoluted with the analysis filters, which comprise a pair of low-pass and high-pass filters. FIG. 13 shows a signal convoluted with the low-pass filter, e.g. 1108a, which has some high frequency components removed. The drastic drop 1302 at the end of the graph is due to the non-overlapping area between the signal and the impulse response of the filter.

FIG. 14 shows the signal after undergoing high-pass filtering. The signal hovers around the x-axis as the zero-frequency component is removed. Having obtained the filtered signal, the redundant sample removal is deemed unnecessary and thus no sub-sampling is performed. When convolution is applied onto the decomposed signal and filters' impulse response, the convoluted signal will be longer than original. Therefore, some samples are trimmed off from both ends of the signal. As the decomposition level increases, the filter's impulse response is zero-padded, where “0”s are inserted between adjacent samples. The zero padding can be useful in reconstructing the wavelet.

In the example embodiments, autocorrelation techniques can be implemented to discover the presence of periodic components within any signal. Autocorrelation is the cross-correlation with shifted versions of the reference signal and a measure of similarity between observations which are shifted in the time domain and is given by equation show below:

Rₓₓ(τ)=∫_{-∞}^{∞}x(t)x(t+τ)dt

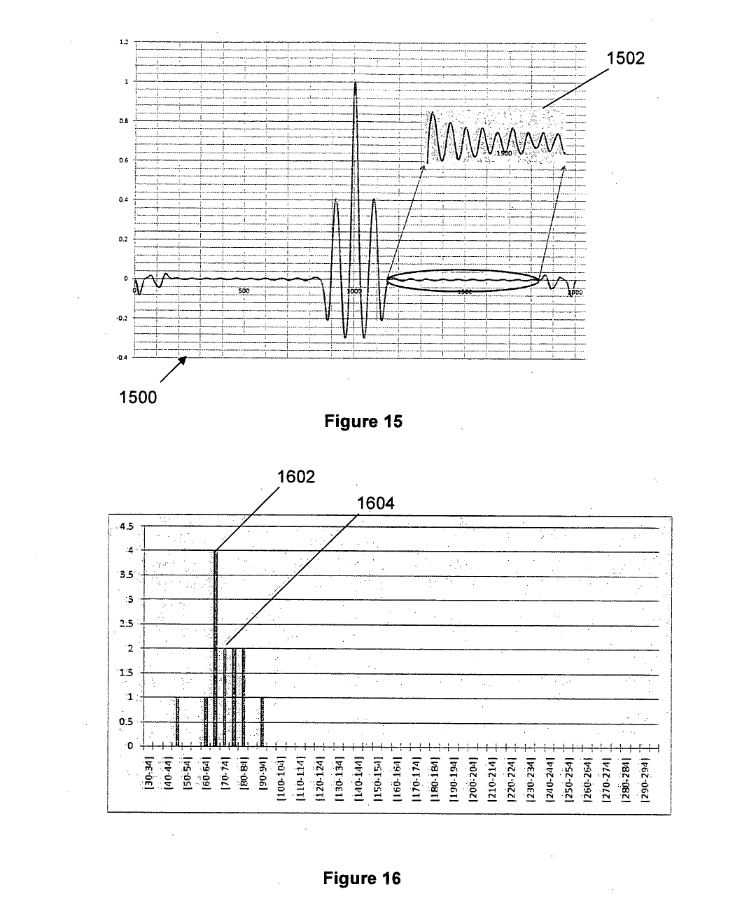

For respiratory and pulse signals, even after wavelet decomposition (denoising), there may still be random noise due to the intensity of small movements in the bed. Through an autocorrelation process, the more periodic respiratory and pulse signals can be enhanced while attenuating the more random noise. In the example embodiments, an auto-correlation is performed on the 5^{th} decomposed signal, 1500 as illustrated in FIG. 15. A sample portion of the auto-correlation output is shown in the zoomed-in portion 1502.

Based on the auto-correlation function, the respiration rate can be derived by studying a histogram of the auto-correlation function. Pulse/heart rates may also be derived in a similar manner, with minor adjustments made to cater to the relatively higher frequency of pulse rates, as would be understood by a person skilled in the art in the context of this description. Firstly, the positive triggered x-axis intersections are tracked down. Thereafter, the time intervals (in terms of sample delays) between each trigger are computed and tabulated as a histogram. FIG. 16 shows a histogram of periodic components obtained from an example embodiment.

From the histogram, the analyser will search for the time interval with the highest occurrence 1602. To prevent any result bias, the analysis further includes an interval adjacent to the interval of highest occurrence with the higher count e.g. 1604. Since the time intervals span over 5 delays, the median value will be considered. The respiratory rate can then be computed by the following equations:

$$Period = \frac{( {count}_{1} * {median}_{1} ) + ( {count}_{2} * {median}_{2} )}{{count}_{1} + {count}_{2}}$$

where count1, median1 belong to the interval with the highest occurrence, e.g. 1602, and count2, median2 belong to the adjacent interval with the higher count e.g. 1604.

As each sample delay is inversely proportional to the sampling rate (sample delay=1/sampling rate), the sampling rate can be used to convert the period into real-time representation. Finally, the result is multiplied by 60 to convert into the standard unit (bpm):

$$Rate = \frac{Sampling Rate * 60}{Period}$$

Returning to FIG. 7, with the completion of step 716, the method proceeds to step 718, where the result is further analysed using Pearson Correlation Coefficients and Logic. Many parameters e.g. the mean, shape, pressure points, histogram, movement index, left right movement, EDSNR, heart rate, respiratory rate and occupancy have been obtained from previous steps, e.g. steps 706, 710 and 716. To enhance robustness of the calculated heart rate or respiratory rate before it is displayed at step 724, further analysis and cross validation can be performed using Pearson Correlation Coefficient.

Using Pearson correlation coefficient, anomalies in the relationship of the parameters can be detected. For example, it is known that there is a direct relationship between respiratory rate and the movement index. One can therefore determine the plausibility of a reading by calculating the covariance and correlation of respiratory rate and movement index.

A rules based engine can also be implemented to determine the state of the monitoring system using simple rule-based logic reasoning based on a DROOLS engine. For example, the system may be configured to send an alert to the caregiver when it is determined that the bed is not occupied, as seen in step 720. As an example, the following algorithm/rule may be used to determine that the bed is not occupied and to trigger the alert.

```
Rule “Unoccupied”
  when
#condition
  pressureFeature(mean < 10.0, histogram delta > 2.0, shape
  similarity<1.0)
  occupancy: OccupancyByPatient( )
then
  occupancy.setUnoccupiedState( );
  system.out.Configured(“Re-Initialized”);
  system.out.SendMessage(“No one on Bed”);
end
```

The robustness of the system can be further enhanced through the integration of e.g. patient history/profile data 726 or through contexts from other modality 728 (such as proximity PIR (Passive InfraRed) sensor which can detect presence of a human patient). Using the rule-based engine, integration of such further data e.g. 726, 728 can be implemented to further enhance the recognition rate of the system and the robustness/accuracy of the data.

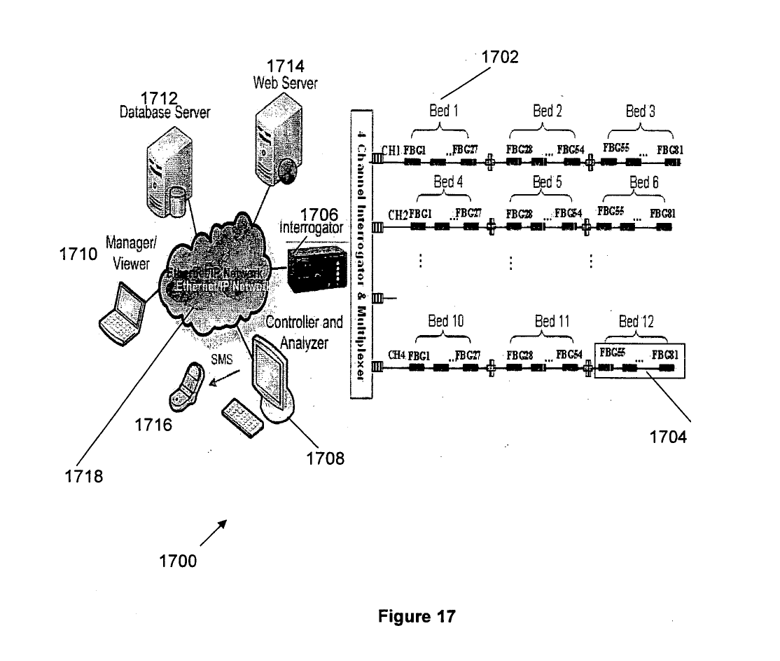

FIG. 17 shows another example embodiment of the present invention 1700 for deployment in a hospital ward. It will be appreciated that the number of sensors per bed is not restricted to 12. The number of sensors deployed may vary depending on the type of mattress and bed being used. It will also be appreciated that the number of beds to be monitored may also vary and the system is flexible and can change according to the number of channels provided and the type of multiplexer used. In this example embodiment, a total of 12 beds e.g. 1702 are monitored, wherein each bed is equipped with 27 FBG sensors e.g. 1704 for monitoring. The Integrator 1706 may be connected to the controller/analyser 1708 via a Ethernet/IP network 1718. It will be appreciated that the controller/analyser 1708 functions similarly to the processing unit 106 in FIG. 1.

In the example embodiment, the controller/analyser 1708 may be connected to a remote manager/viewer 1710, which can allow for the access of the status of any bed to be viewed or controlled remotely over the Ethernet/IP network 1718. Similarly, data such as patient history, stored in a remote database server 1712 and/or a web server 1714, may be accessible via the Ethernet/IP network 1718. The controller 1708 may also be connected to the GSM network such that it can send text messages via SMS to intended recipients e.g. doctors or nurses in the event of emergencies such as when the patient is not in his bed etc.

In the example embodiment illustrated in FIG. 4, the sensors 402 are placed on the frame of the bed, but beneath the mattress placed on the frame of the bed. In addition to the sensor array illustrated FIG. 4, alternatively or additionally, a sensor array may be placed above the mattress. For example, sensors placed beneath the mattress (or bottom-layer sensors) may be used for observing the pressure profile and occupancy of the patient, while sensors placed on the mattress (or top-layer sensors) may be used for e.g. respiratory and pulse rate monitoring for improved accuracy. The top-layer sensors may be connected to the bottom-layer sensors through a connector with one end of the sensors connected to the fiber wire laid on the wall of the ward, for further connection with the e.g. interrogator.

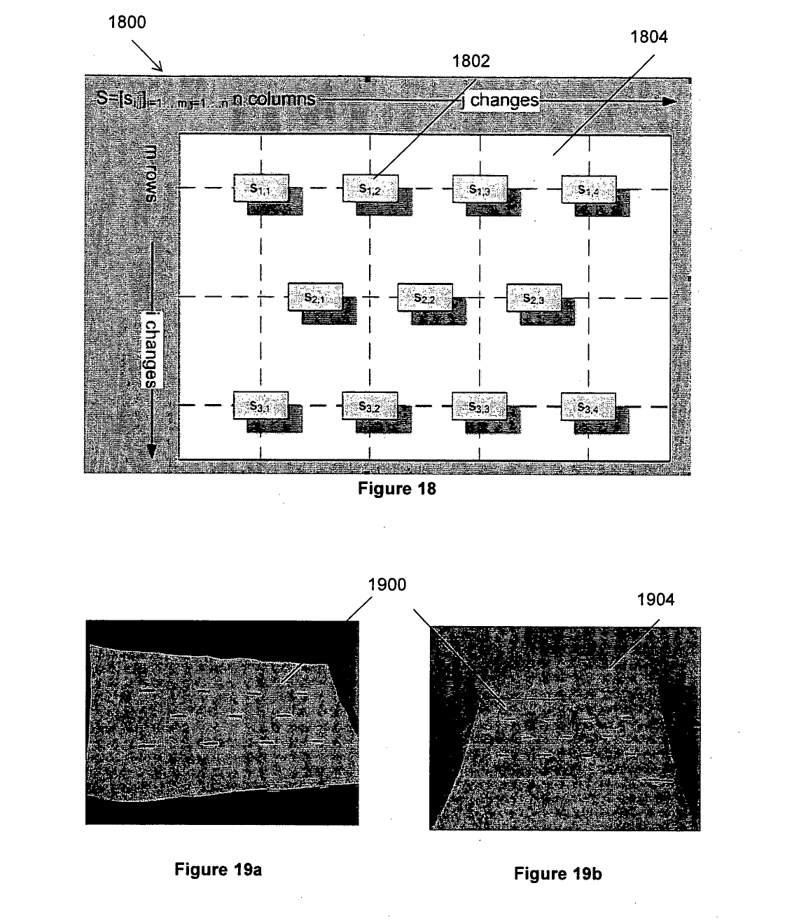

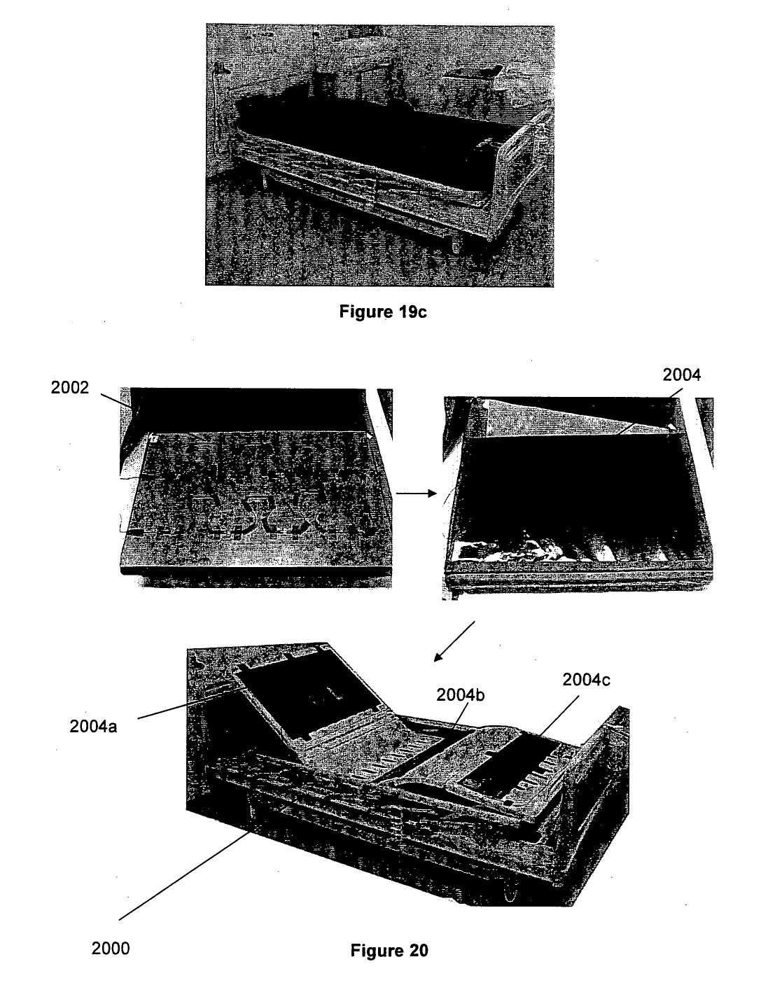

FIG. 18 shows an example embodiment of a sensor array 1800 of sensors e.g. 1802 paced on top of a mattress 1804. The sensors 1802, are carefully positioned to cover the full width of the bed such that vital signals can be detected even if the patient change their lying positions. FIG. 19a shows an example implementation 1900 of the sensor array. The sensor array 1900 is placed near an approximated chest area of a patient and fastened to the mattress 1904 as shown in FIG. 19b. As further shown in FIG. 19c, bed sheets may be placed over the mattress 1902, such that the sensor array 1900 is not visible.

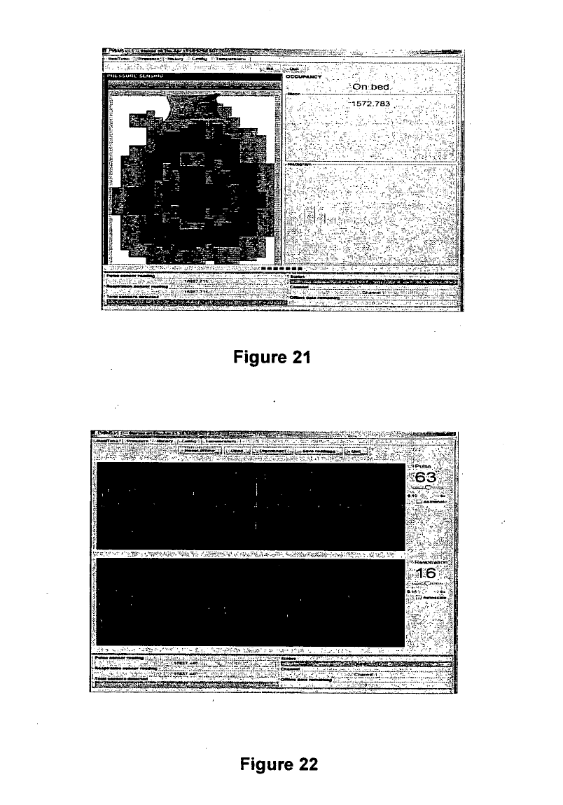

It will be appreciated that modern hospital bed frames are flexible and can be adjusted into numerous configurations, to allow for a patient lying on top of the bed to be moved accordingly. FIG. 20 shows an example embodiment of an adjustable bed frame 2000. In the example embodiment, the sensors e.g. 2002 placed on top of the bed frame are packaged into three different sections 2004a, 2004b, 2004c to fit the adjustable bed frame 1900, catering to the movements of the different sections of the bed frame.

FIG. 21 shows a snapshot of automated pressure profile and occupancy monitoring provided by an example embodiment of the present invention. FIG. 22 shows a snapshot of automated respiratory and pulse rate monitoring provided by an example embodiment of the present invention.

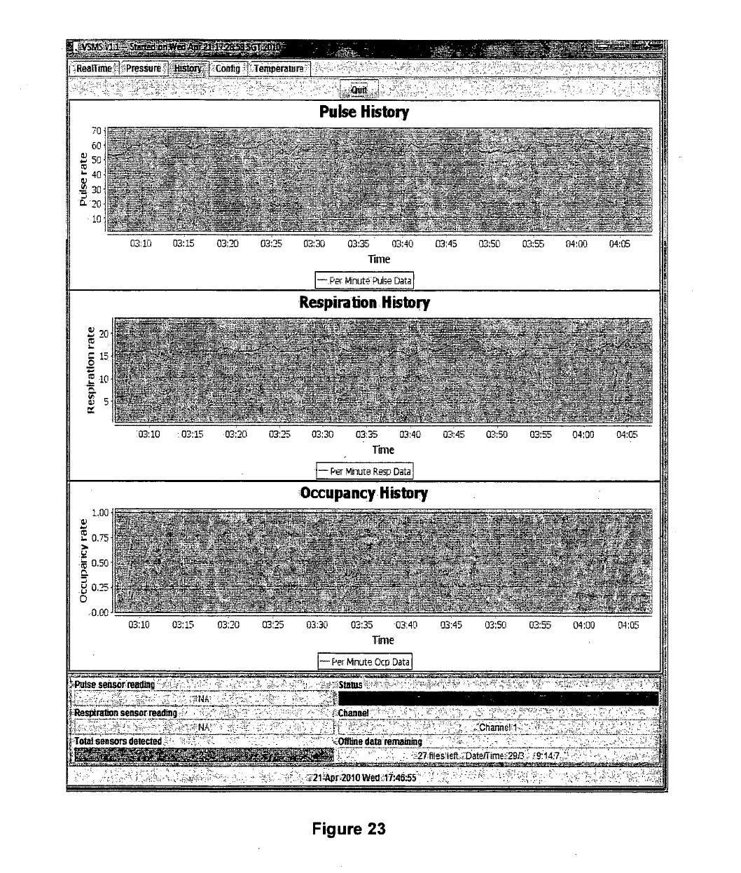

Embodiments of the present invention seek to provide a continuous and non-intrusive approach to monitor respiratory rate, heart rate, pressure points and occupancy of patient on a bed in a robust manner. It will be appreciated that with continuous monitoring, historical and trend charts may be plotted as shown in FIG. 23, which can be valuable to doctors for diagnosis. Periodic but infrequent checks performed by systems of the prior art are not continuous and may therefore miss the onset of crisis events.

The embodiments of the present invention also utilise a plurality of processing techniques which can remove noisy signals due to small and large user's movement and provides feedback based on Euclidean Distance SNR (EDSNR) for sensor selection and configuration within a sensor array for robust monitoring, which can significantly reduce the false alarm rate. Context information from the user or acquired through other modality can also be used to fine tune the system to enhance the overall recognition rate.

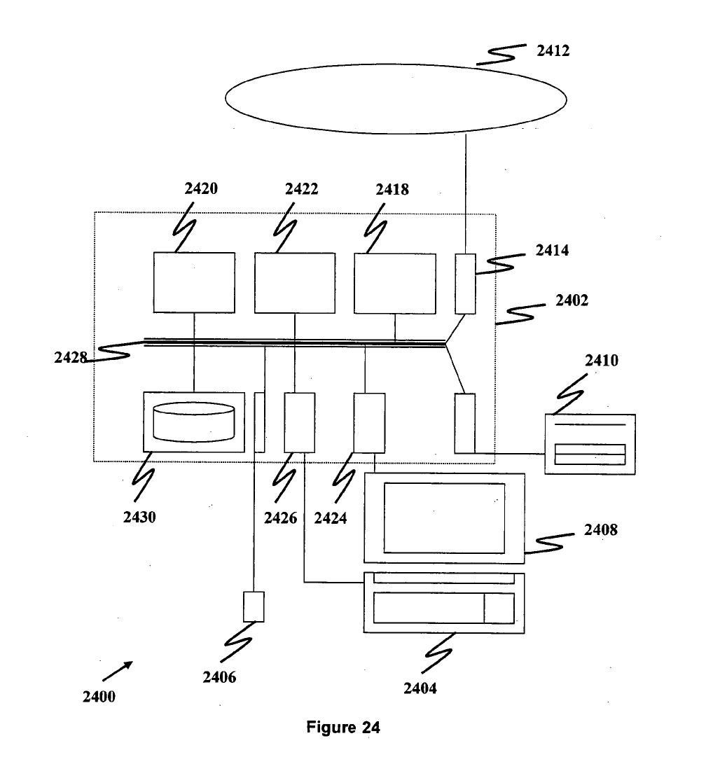

The method and system of the example embodiment can be implemented on a computer system 2400, schematically shown in FIG. 24. It may be implemented as software, such as a computer program being executed within the computer system 2400, and instructing the computer system 2400 to conduct the method of the example embodiment.

The computer system 2400 comprises a computer module 2402, input modules such as a keyboard 2404 and mouse 2406 and a plurality of output devices such as a display 2408, and printer 2410.

The computer module 2402 is connected to a computer network 2412 via a suitable transceiver device 2414, to enable access to e.g. the Internet or other network systems such as Local Area Network (LAN) or Wide Area Network (WAN).

The computer module 2402 in the example includes a processor 2418, a Random Access Memory (RAM) 2420 and a Read Only Memory (ROM) 2422. The computer module 2402 also includes a number of Input/Output (I/O) interfaces, for example I/O interface 2424 to the display 2408, and I/O interface 2426 to the keyboard 2404.

The components of the computer module 2402 typically communicate via an interconnected bus 2428 and in a manner known to the person skilled in the relevant art.

The application program is typically supplied to the user of the computer system 2400 encoded on a data storage medium such as a CD-ROM or flash memory carrier and read utilising a corresponding data storage medium drive of a data storage device 2430. The application program is read and controlled in its execution by the processor 2418. Intermediate storage of program data maybe accomplished using RAM 2420.

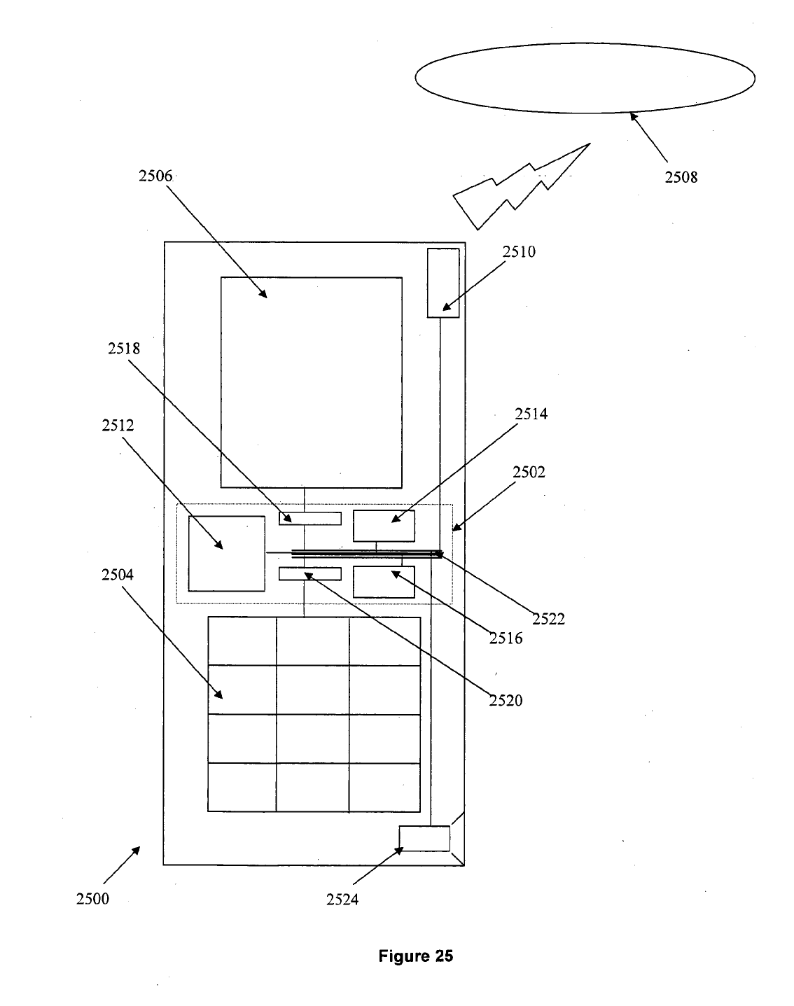

The method of the current arrangement can be implemented on a wireless device 2500, schematically shown in FIG. 25. It may be implemented as software, such as a computer program being executed within the wireless device 2500, and instructing the wireless device 2500 to conduct the method.

The wireless device 2500 comprises a processor module 2502, an input module such as a keypad 2504 and an output module such as a display 2506.

The processor module 2502 is connected to a wireless network 2508 via a suitable transceiver device 2510, to enable wireless communication and/or access to e.g. the Internet or other network systems such as Local Area Network (LAN), Wireless Personal Area Network (WPAN) or Wide Area Network (WAN).

The processor module 2502 in the example includes a processor 2512, a Random Access Memory (RAM) 2514 and a Read Only Memory (ROM) 2516. The processor module 2502 also includes a number of Input/Output (I/O) interfaces, for example I/O interface 2518 to the display 2506, and I/O interface 2520 to the keypad 2504.

The components of the processor module 2502 typically communicate via an interconnected bus 2522 and in a manner known to the person skilled in the relevant art.

The application program is typically supplied to the user of the wireless device 2500 encoded on a data storage medium such as a flash memory module or memory card/stick and read utilising a corresponding memory reader-writer of a data storage device 2524. The application program is read and controlled in its execution by the processor 2512. Intermediate storage of program data may be accomplished using RAM 2514.

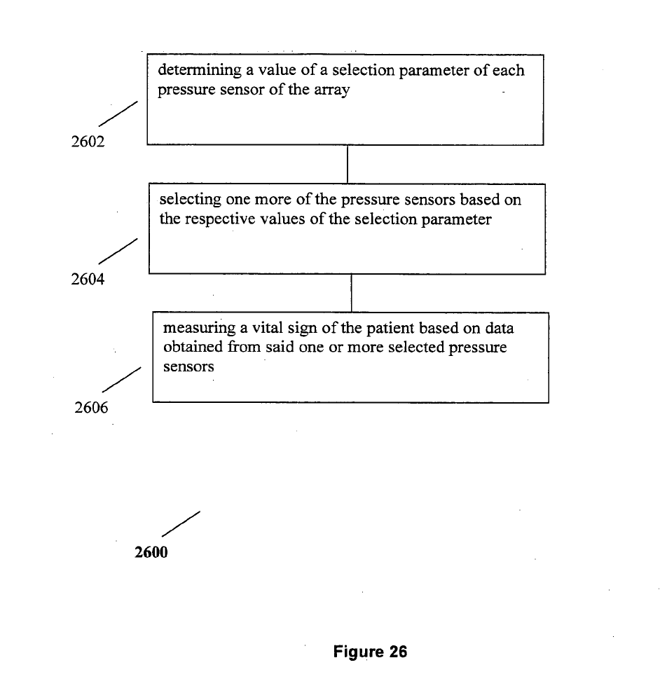

FIG. 26 is a flow chart 2600 illustrating a method of patient monitoring using an array of pressure sensors in an example embodiment. At step 2602, a value of a selection parameter of each pressure sensor of the array is determined. At step 2604, one more of the pressure sensors is selected based on the respective values of the selection parameter. At step 2606, a vital sign of the patient is measured based on data obtained from said one or more selected pressure sensors.

It will be appreciated by a person skilled in the art that numerous variations and/or modifications may be made to the present invention as shown in the specific embodiments without departing from the spirit or scope of the invention as broadly described. The present embodiments are, therefore, to be considered in all respects to be illustrative and not restrictive.

It will be appreciated by a person skilled in the art that while the example embodiments show the use of FBG optical sensors, other sensors e.g. electrical sensors, intensity-based optical sensors, distributed reflectometry optical sensors may also be used.

1. A method for monitoring a patient using an array of pressure sensors, the method comprising the steps of:
determining a value of a selection parameter of each pressure sensor of the array;
selecting one more of the pressure sensors based on the respective values of the selection parameter; and
measuring a vital sign of the patient based on data obtained from said one or more selected pressure sensors.

2. The method as claimed in claim 1, wherein the determining a value of a selection parameter comprises:
determining a desired sensor location; and
determining a distance of each pressure sensor from the desired sensor location.

3. The method as claimed in claim 2, comprising choosing a default pressure sensor as the selected pressure sensor when a distance between the default pressure sensor and the desired sensor location is within a threshold.

4. The method as claimed in claim 2, comprising choosing another one of the pressure sensors as the selected pressure sensor when a distance between the default pressure sensor and the desired sensor location is outside a threshold

5. The method as claimed in any one of the claim 2, wherein the step of determining the desired sensor location comprises the steps of;
approximating a shape of the patient based on data from the pressure sensors; and
determining the desired sensor location based on the determined shape.

6. The method as claimed in claim 1, further comprising determining a presence of the patient based on data from the pressure sensors.

7. The method as claimed in claim 6, wherein the step of determining the presence of the patient comprises performing one or more of a group consisting of mean, histogram, and shape analysis.

8. The method as claimed in claim 1, further comprising determining a movement of the patient based on data from the pressure sensors.

9. The method as claimed in claim 8, wherein the step of determining a movement of the patient on the surface comprises one or more of a group consisting of conducting pressure point analysis using regression techniques and conducting peak detection techniques.

10. The method as claimed in claim 1, wherein the vital sign comprises heart rate or respiratory rate.

11. The method as claimed in claim 1, wherein determining the vital sign comprises one or more of a group consisting of wavelet denoising, autocorrelation and histogram techniques.

12. The method as claimed in claim 1, further comprising analyzing the vital sign result with Pearson correlation coefficients.

13. The method as claimed in claim 1, further comprising analyzing the vital sign result with integrated patient information or other contexts.

14. The method as claimed in claim 1, further comprising configuring an output response in response to the vital sign result.

15. A system for monitoring a patient using an array of pressure sensors, the system comprising:
means for determining a value of a selection parameter of each pressure sensor of the array;
means for selecting one more of the pressure sensors based on the respective values of the selection parameter; and
means for measuring a vital sign of the patient based on data obtained from said one or more selected pressure sensors.

16. The system as claimed in claim 15, wherein the means for determining a value of a selection parameter comprises:
means for determining a desired sensor location; and
means for determining a distance of each pressure sensor from the desired sensor location.

17. The system as claimed in claim 16, wherein a default pressure sensor is chosen as the selected pressure sensor when a distance between the default pressure sensor and the desired sensor location is within a threshold.

18. The system as claimed in claim 16, wherein another one of the pressure sensors is chosen as the selected pressure sensor when a distance between the default pressure sensor and the desired sensor location is outside a threshold

19. The system as claimed in claim 16, wherein the means for determining the desired sensor location comprises;
means for approximating a shape of the patient based on data from the pressure sensors; and
means for determining the desired sensor location based on the determined shape.

20. The system as claimed in claim 1, further comprising means for determining a presence of the patient based on data from the pressure sensors.

21. The system as claimed in claim 20, wherein the means for determining the presence of the patient comprises means for performing one or more of a group consisting of mean, histogram, and shape analysis.

22. The system as claimed in claim 15, further comprising means for determining a movement of the patient based on data from the pressure sensors.

23. The system as claimed in claim 22, wherein the means for determining a movement of the patient on the surface comprises one or more of a group consisting of means for conducting pressure point analysis using regression techniques and means for conducting peak detection techniques.

24. The system as claimed in claim 15, wherein the vital sign comprises heart rate or respiratory rate.

25. The system as claimed in claim 15, wherein means for determining the vital sign comprises one or more of a group consisting of mean for wavelet denoising, autocorrelation and histogram techniques.

26. The system as claimed in claim 15, further comprising means for analyzing the vital sign result with Pearson correlation coefficients.

27. The system as claimed in claim 15, further comprising means for analyzing the vital sign result with integrated patient information or other contexts.

28. The system as claimed in claim 15, further comprising means for configuring an output response in response to the vital sign result.

29. A computer readable data storage medium having stored thereon computer code means for instructing a computer to execute a method for monitoring a patient using an array of pressure sensors, the method comprising the steps of:
determining a value of a selection parameter of each pressure sensor of the array;
selecting one more of the pressure sensors based on the respective values of the selection parameter; and
measuring a vital sign of the patient based on data obtained from said one or more selected pressure sensors.
